(19) United States
(12) Patent Application Publication
(10) Pub. No.: US 2023/0078870 A1
(43) Pub. Date: Mar. 16, 2023

(54) SYSTEMS AND METHODS FOR DETERMINING A STERILIZATION TIME AND STERILIZING A VEHICLE INTERIOR

(71) Applicant: Honda Motor Co., Ltd., Tokyo (JP)

(72) Inventors: David M. Kirsch, Torrance, CA (US); Jerry Erin Bonnah, Huntington Beach, CA (US)

(21) Appl. No.: 17/476,965

(22) Filed: Sep. 16, 2021

(51) Int. Cl. A61L 2/10

(52) U.S. Cl. CPC A61L 2/10; A61L 2202/11; A61L 2202/14; A61L 2202/25

(57) ABSTRACT

A system for determining a sterilization time includes, at least, a memory device and a processor. The processor is configured to receive, from at least one detector device located within the vehicle interior, a first intensity of ultraviolet (UV) energy from sunlight incident on the at least one detector device. In addition, the processor is configured to determine, based at least in part on the first intensity of UV energy received from the detector device, a sterilization time for sterilizing the vehicle interior. The processor is also configured to transmit the sterilization time to a user device, which may display at least the sterilization time and/or a progress toward sterilization.

### BACKGROUND

The present disclosure relates generally to vehicle sterilization and, more specifically, to systems and methods for determining a sterilization time and sterilizing a vehicle interior based upon the determined sterilization time.

At least some known systems for use in sanitizing an object use chemical agents, radiation, such as ultraviolet light, and/or other anti-pathogens to destroy virus particles, bacteria, and other potentially harmful substances residing on the object. However, many conventional sanitizing systems are not designed for, and/or are not convenient for, use in sanitizing a vehicle interior. In addition, to the extent that systems for vehicle sanitization have been developed (e.g., in a post-COVID era), sanitizing processes do not typically account for naturally-occurring anti-pathogens, such as sunlight. Likewise, conventional sanitizing systems fail to provide a user with any sort of progress indication that represents an elapsed amount of progress toward the full completion of a sanitizing process and/or an amount time remaining to fully complete the sterilization process.

Accordingly, it would be desirable to have effective systems and methods for use in sterilizing and/or sanitizing objects, including vehicle interiors. More particularly, systems and methods that account for naturally-occurring anti-pathogens, such as sunlight, in producing an estimated sterilization time are desirable. Moreover, systems and methods that provide one or more indicia of an elapsed progress toward sterilization are also desirable.

### BRIEF DESCRIPTION

In one aspect, a system for determining a sterilization time for sterilizing an interior of a vehicle is described. The system includes a memory device and a processor. The processor is configured to receive, from at least one detector device located within the vehicle interior, a first intensity of ultraviolet (UV) energy from sunlight incident on the at least one detector device. In addition, the processor is configured to determine, based at least in part on the first intensity of UV energy received from the detector device, a sterilization time for sterilizing the vehicle interior. The processor is also configured to transmit the sterilization time to a user device, which may display at least the sterilization time and/or a progress toward sterilization.

In another aspect, a system for sterilizing an interior of a vehicle is described. The system includes at least one ultraviolet (UV) detector device disposed within the vehicle interior, at least one powered UV source device disposed within the vehicle interior, a memory device, and a processor. The processor is configured to receive, from at least one detector device located within the vehicle interior, a first intensity of ultraviolet (UV) energy from sunlight incident on the at least one detector device. In addition, the processor is configured to determine, based at least in part on the first intensity of UV energy received from the detector device, a sterilization time for sterilizing the vehicle interior. The processor is also configured to control the powered UV source device to emit UV energy within the vehicle interior for the sterilization time.

In yet another aspect, a method for determining a sterilization time for a vehicle interior is described. The method includes receiving, by a processor and from at least one detector device located within the vehicle interior, a first intensity of ultraviolet (UV) energy from sunlight incident on the at least one detector device. The method also includes determining, by the processor and based at least in part on the first intensity of UV energy received from the detector device and a second intensity of UV energy emitted by a powered UV source device located within the vehicle interior, a sterilization time for sterilizing the vehicle interior. In addition, the method includes providing, by the processor, the sterilization time to a user device, wherein the user device is configured to display one of the sterilization time or a progress toward sterilizing the vehicle interior.

### BRIEF DESCRIPTION OF THE DRAWINGS

FIG. 1 is a block diagram of an exemplary system for use in sterilizing, or disinfecting, an interior of a vehicle.

FIG. 2 illustrates a portion of an exemplary reference lookup table that may be used by the system shown in FIG. 1 to determine a time for a full sterilization based on one or more sterilization factors.

FIG. 3 is a front view of an exemplary user device, such as a smartphone, that may be used with the system shown in FIG. 1 to display information related to sterilization of a vehicle interior.

FIG. 4 is a flowchart illustrating an exemplary process that may be implemented for determining a sterilization time using the system shown in FIG. 1.

### DETAILED DESCRIPTION

Systems and methods for use in determining an amount of time for sterilization, i.e., a sterilization time, and systems and methods for use in sterilizing an interior of a vehicle based on the determined sterilization time are also described herein. In at least some implementations, one or more sterilization factors, such as an amount of a first UV energy received from sunlight, an amount of a second UV energy received from a powered UV source device, an elapsed time since the vehicle was last occupied, and/or an internal temperature of the vehicle interior may be analyzed to determine the sterilization time. As a result, at least some environmental factors, such as the amount of UV from sunlight, the elapsed time since the vehicle was last occupied, and the internal temperature of the vehicle may be factored into the determination of an appropriate sterilization time. As such, any increase in any of the aforementioned environmental factors may generally tend to reduce a sterilization time, during which the powered UV source device is energized to irradiate the vehicle interior with UV light. Moreover, the sterilization time may be communicated to a user device, such as a smartphone, and/or may be calculated by the user device itself, to facilitate display of the sterilization time and/or a variety of other status indicators for the convenience of the user. In at least some embodiments, the vehicle may be locked or otherwise secured to prevent entry by the user and/or other intended occupants during the sterilization process and until the sterilization time has elapsed. In addition, although the systems described herein are illustrated in association with a motor vehicle, the disclosure is also intended for use with any means of conveyance including boats, aircraft, stationary objects, and other vehicles and objects capable of human occupation or human interaction.

As used herein, the terms “sterilize,” “sterilizing,” “sterilization,” “disinfect,” “disinfecting,” “sanitize,” and “sanitization” generally refer to the removal and/or destruction, such as by the application of ultraviolet light, to a pathogen, such as a virus, a bacteria, and/or any other pathogen or material that may be harmful to human health. It will be appreciated that these terms may be used to mean removal or destruction of pathogens within a predefined threshold level, such as for example, removal of 99% of pathogens, 99.9% of pathogens, and the like. As a result, in general, these terms do not necessarily imply the eradication of 100% of a given pathogen from the surface of an object, although in at least some cases, the systems and methods described herein may achieve such a result.

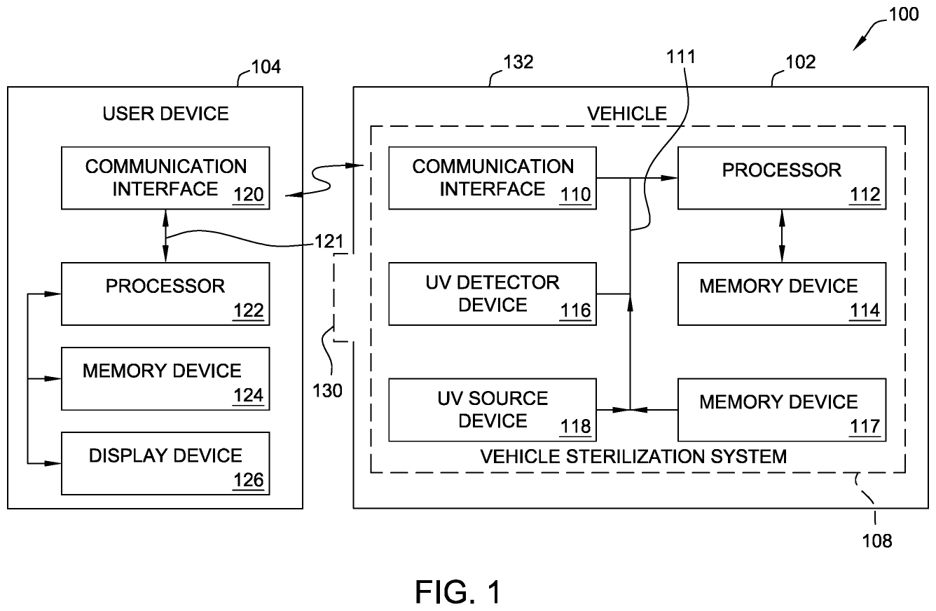

FIG. 1 is a block diagram of an exemplary vehicle 102 and an exemplary user device 104. User device 104 may include any of a variety of suitable user devices, such as a smartphone, a tablet, and the like. Vehicle 102 may include any passenger vehicle, such as any motor vehicle, any boat or aircraft, any commercial or industrial vehicle or machine, and/or any other object or mode of transportation, means of conveyance, or object that can be occupied by a human and/or by an animal.

In the exemplary embodiment, vehicle 102 includes a vehicle interior 106, such as an area typically occupied by a driver and/or at least one passenger. Vehicle 102 also includes a vehicle sterilization system 108, which may be used to selectively sterilize vehicle interior 106, and which may provide information, as described herein, to user device 104.

In at least some implementations, vehicle sterilization system 108 includes a communication interface 110, a processor 112, a memory device 114, an ultraviolet (“UV”) detector device 116, a vehicle safety system 117, and/or a powered UV source device 118, any of which, alone or in combination, may be communicatively coupled for communication with processor 112, such as by a system bus 111. In the exemplary embodiment, communication interface 110 may include any wireless communication interface that enables wireless communication with user device 104. For example, communication interface 110 may be a radio frequency (“RF”) transceiver enabled for a communication protocol, such as Bluetooth, WiFi, cellular 3G/4G/5G, and/or any other suitable protocol.

In addition, detector device 116 may be any UV detector, such as any narrowband radiometer, broadband radiometer, a spectrophotometer, and/or any other device or photodetector that detects intensity and/or amount of UV energy incident thereon.

Source device 118 may include any device that is capable of producing and/or emitting UV energy. For example, source device 118 may include an electrically powered UV lamp. In some embodiments, source device 118 may output UV energy in a range of 1.0-100,000 mW/cm². However, it should be appreciated that other ranges are contemplated by and within the scope of the present disclosure. In addition, source device 118 may be selected to have an output that facilitates improving, optimizing, and/or reducing a sterilization time, as described herein. For example, in general terms, a sterilization time may be reduced by increasing the UV energy output from source device 118. Accordingly, in at least one embodiment, a high value of UV output (e.g., up to 100,000 mW/cm²) may be selected to “rapidly” sterilize (or “flash sterilize”) vehicle interior

Although a single detector device 116 and a single source device 118 are illustrated in FIG. 1, it will be appreciated that any suitable number of detector devices 116 and/or source devices 118 may be included within vehicle interior 106. For example, in at least one embodiment, a plurality of source devices 118 sufficient to irradiate all, or a desired portion, of vehicle interior 106 may be included in sterilization system 100.

In the exemplary implementation, user device 104 includes a communication interface 120, a processor 122, a memory device 124, and/or a display device 126, any of which, alone or in combination, may be coupled for communication with processor 112, such as via a system bus 121. Communication interface 120 may include any wireless communication interface that enables wireless communication with vehicle sterilization system 108. For example, communication interface 120 may be a radio frequency (“RF”) transceiver enabled for a communication protocol, such as Bluetooth, WiFi, cellular 3G/4G/5G, and/or any other suitable protocol. Display device 126 may include any of a variety of display devices, such as any liquid crystal display (“LCD”) device, any touchscreen display device, such as any capacitive touchscreen display device, and the like. More generally, as described herein, user device 104 may be a smartphone, a tablet, and/or any other suitable personal computing device.

In operation, detector device 116 may measure an intensity and/or an amount of UV energy incident thereon, such as in units of mW/cm². The UV energy incident on detector device 116 may include natural light, such as sunlight. As a result, detector device 116 may be positioned within vehicle interior 106 to measure an intensity or amount of UV energy from sunlight. In some embodiments, detector device 116 may be shielded from UV light emitted by UV source device 118. For example, detector device 116 may be positioned within vehicle interior 106 and enclosed within a housing (not shown) that facilitates substantially shielding and thus preventing UV light emitted by UV source device 118 from being detected or measured by detector device 116. As such, detector device 116 may, in at least some implementations, only measure and be impacted by UV passing through a window 130 of vehicle 102. In another embodiment, detector device 116 may be located on an exterior surface 132 of vehicle 102. In yet another embodiment, detector device 116 may not be shielded from UV energy emitted by UV source device 118. Rather, in at least one embodiment, detector device 116 may measure the intensity or amount of UV energy from natural sunlight prior to any UV energy being emitted by UV source device 118.

In the exemplary embodiment, processor 112 may receive a value of the measured intensity from detector device 116, such as via system bus 111. In addition, processor 112 may determine a value of the intensity of UV energy emitted by source device 118 when source device 118 is operating (e.g., a power rating of source device 118). To obtain the energy emitted by source device 118, processor 112 may query or otherwise receive the value from source device 118, such as in real-time. In another embodiment, processor 112 may retrieve the value from memory device 114, which may store one or more predefined settings for source device 118. In such an embodiment, processor 112 may also retrieve a predefined setting from memory device 114 to control the UV energy emitted by source device 118. As a result, in at least some embodiments, UV source device 118 can emit UV light at different selectable and variable intensities, and can be selectively controlled, via processor 112 and/or by any device, including user device 104, that is in communication with vehicle sterilization system 108, to irradiate vehicle interior 106 at different UV intensities. Further, in at least some embodiments, UV source device 118 may be capable of selectively emitting UV energy in different portions of the UV spectrum, such as UVA, UVB, and/or UVC. Processor 112 may be operable to selectively control UV source device 118 to emit UV energy in one more of these spectra. In addition, the measured UV intensities obtained by processor 112 may, in at least some embodiments, be wirelessly transmitted (in addition to one or more other sterilization factors 202-208, as described below), to processor 122 of user device 104.

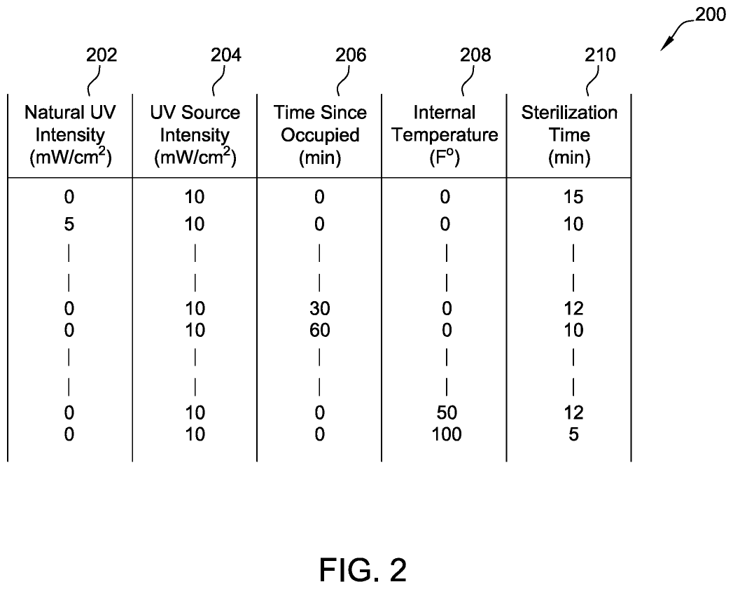

FIG. 2 illustrates a portion of an exemplary lookup table 200 that may be used by vehicle sterilization system 100 (shown in FIG. 1) to determine a sterilization time 210 based on one or more sterilization factors 202-208. Sterilization factors 202-208, described in more detail below, may include, but are not limited to any of: i) an amount of first UV energy 202 from sunlight (e.g., as measured by detector device 116), ii) an amount of second UV energy 204 output by source device 118, iii) an elapsed amount of time 206 since vehicle 102 was last occupied, iv) an internal temperature 208 of vehicle interior 106, and/or the like.

Accordingly, in at least some implementations, processor 112 and/or processor 122 may analyze one or more sterilization factors 202-208 to determine a sterilization time 210. More particularly, in some embodiments, vehicle sterilization system 108 may determine sterilization time 210. In other embodiments, user device 104 may determine sterilization time 210. In the exemplary embodiment, and as described in additional detail herein, processor 112 determines sterilization time 210 and transmits sterilization time 210 to processor 122 of user device 104 (e.g., via communications interfaces 110 and 120) for display by user device 104. However, in at least some embodiments, processor 112 may transmit sterilization factors to processor 122 to enable calculation of sterilization time 210 by processor 122. In some embodiments, processor 112 determines sterilization time 210 and displays sterilization time 210 and/or any of the other visual indicia described herein on a display device of vehicle 102 (e.g., an in-dash display).

As described herein, sterilization time 210 may be provided, such that, given a predefined combination of sterilization factors 202-208, as shown in lookup table 200, a threshold amount of sterilization or disinfection may be achieved within a corresponding sterilization time 210. For example, the various combinations of sterilization factors 202-208 shown in table 200 may be provided to achieve 95% sterilization, 98% sterilization, 99% sterilization, 99.9% sterilization, and/or any other suitable level of disinfection, within the sterilization time 210 that corresponds to each respective combination of factors 202-208.

As a result, and in general terms, vehicle sterilization system 108 and/or user device 104 may analyze the sterilization factors 202-208, as described herein, to determine a sterilization time 210, which may be provided in different visual presentation modes to a user. Thus as a result, the user may receive an estimate or indication of a progress towards sterilization, a time remaining until sterilization is complete, and/or the like.

More particularly, in at least one example, and as shown with continuing reference to FIG. 2, an amount of first UV energy 202 from sunlight may be analyzed in conjunction with one of more other factors, such as any of factors 204-208, to obtain a sterilization time 210. Although specific values are provided in table 200, it will be appreciated that these values are merely exemplary and that many other values may be provided in table 200 without departing from the scope of the present disclosure. Accordingly, the values included in table 200 should not be construed as limiting in any way, but merely as exemplary values intended to better illustrate the systems and methods of the present disclosure.

Accordingly, in the illustrated example, a first UV energy 202 of about 0 mW/cm² (e.g., during non-sunlight hours, such as during the evening hours) and an amount of second UV energy 204 of about 10 mW/cm² may yield a sterilization time 210 of about fifteen minutes. However, if an amount of first UV energy 202 of about 5 mW/cm² (e.g., during daytime hours) and an amount of second UV energy 204 of about 10 mW/cm² may yield a sterilization time 210 of about ten minutes. Thus, the sterilization time 210 may be selectively adjusted depending upon an amount of sunlight present and/or an output of source device 118. That is, in the presence of UV from sunlight, or as an intensity of source device 118 is increased, sterilization times 210 may generally be reduced.

In addition to sterilization factors 202 and 204, one or more other sterilization factors 206-208, such as an elapsed time 206 since vehicle 202 was last occupied by a human, may also be analyzed in conjunction with factors 202, 204, and/or 208 to determine sterilization time 210. For example, as shown in FIG. 2, an elapsed time 206 of about thirty minutes since vehicle 106 was last occupied may result in a sterilization time 210 (all other factors being held constant for simplicity) of about twelve minutes. Likewise, a time 206 of about sixty minutes since vehicle 106 was last occupied may result in a sterilization time 210 of about ten minutes. Stated another way, as the period of time 206 since vehicle 102 was last occupied increases, shorter sterilization times 210 may be provided or required.

Similarly, internal temperature 208 of vehicle interior 106 may also be analyzed in conjunction with one or more of factors 202-206 to determine sterilization time 210. For example, as shown in FIG. 2, an internal temperature 208 of 50° Fahrenheit may result in a sterilization time 210 (with all other factors being held constant, for simplicity) of about twelve minutes. Likewise, an internal temperature 208 of about 100° Fahrenheit may result in a sterilization time 210 of about 5 minutes. Stated another way, as the internal temperature 208 of vehicle 102 increases, shorter sterilization times 210 may be provided or required.

Accordingly, a plurality of sterilization factors 202-208 may be analyzed in different combinations and depending upon one or more sensed environmental factors, such as first UV energy 202, time 206, and/or internal temperature 208, to determine a range of sterilization times 210, which may be influenced by any or all of sterilization factors 202-208, including those that may occur as a result of natural environmental conditions surrounding vehicle 102 (e.g., UV from sunlight, internal temperature, and the like). System 100 may thus account for, in determining an appropriate sterilization time 210, a variety of environmental factors.

In addition to determining sterilization time 210, in at least some embodiments, vehicle sterilization system 108 may initiate a sterilization process within vehicle interior 106. For example, processor 112 may control UV source device 118 (and/or a plurality of UV source devices 118) to irradiate vehicle interior 106 with UV energy. In some embodiments, processor 112 may control vehicle safety system 117 to lock and/or unlock one or more doors and/or windows of vehicle 102, such as to prevent entry and/or access by an occupant during the sterilization of vehicle interior 106.

In at least one exemplary implementation, user device 104 may prompt the user to confirm that vehicle 102 is not occupied (e.g., by humans or animals). In response to receiving a confirmation that vehicle 102 is not occupied, processor 112 may control safety system 117 to lock the doors and/or windows of vehicle 102. In addition, processor 112 may control UV source device 118 to irradiate vehicle interior 106 for the duration of sterilization time 210, and following completion of sterilization time 210, processor 112 may control UV source device 118 to halt irradiation of vehicle interior 106. After UV source device 118 has been controlled to cease operating, i.e, after source device 118 has been de-energized, processor 112 may unlock vehicle 102 for entry by one or more occupants.

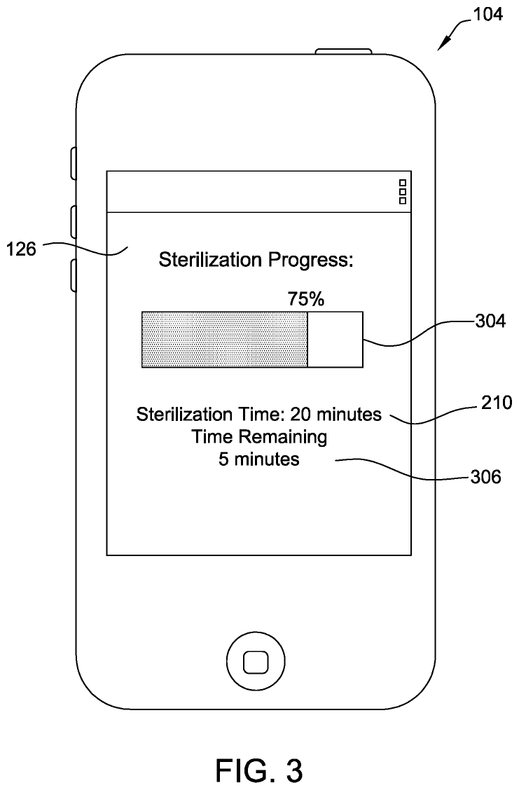

FIG. 3 is a front view of user device 104, such as a smartphone, that displays information related to sterilization of vehicle interior 106. For example, as shown, following determination of an appropriate sterilization time 210 (e.g., using table 200), processor 112 and/or processor 122 may control a display device, such as display device 126 of user device 104, to display the determined sterilization time 210. In at least some embodiments, user device 104 may display a variety of other visual indicia as well, such as a sterilization progress 302, which may be provided in any suitable format, such as by a progress bar 304. In some embodiments, as shown, a countdown timer 306 showing a time remaining until sterilization is complete may also be displayed. Likewise, although not shown, other visual indicia, such as an hourglass indicating that sterilization is occurring and/or another meter, such as a meter that changes from red to green to indicate progress toward sterilization, may be displayed. In addition, as described above, user device 104 may prompt the user to confirm that vehicle 102 is not occupied, such as prior to initiating the sterilization process, to protect the health and safety of any individual who may subsequently wish to occupy vehicle 102.

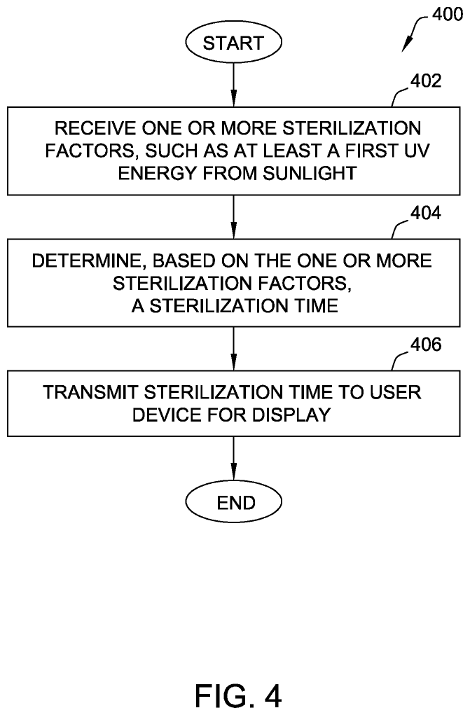

FIG. 4 is a flowchart illustrating an exemplary process 400 for determining a sterilization time and sterilizing vehicle interior 106 based upon the determined sterilization time. Accordingly, as shown, in at least some embodiments, and as described in additional detail above, processor 112 may obtain one or more sterilization factors, such as one or more of i) first UV energy 202 from sunlight (e.g., as measured by detector device 116), ii) second UV energy 204 output by source device 118, iii) time 206 since vehicle 102 was last occupied, iv) internal temperature 208 of vehicle interior 106, and the like (step 402). In addition, processor 112 may determine, based on the one or more sterilization factors 202-208, sterilization time 210 (step 404). Moreover, in response to determining sterilization time 210, processor 112 may transmit, such as via a wireless communication protocol, as described herein, sterilization time to processor 122 of user device 104 (step 404). In response to receiving sterilization time 210, processor 122 may control a display device, such as a display device 126 of user device 104, to display sterilization time 210, a progress or status bar 306 indicating a progress toward sterilization, a countdown timer 306 indicating a time remaining to completion of a sterilization or irradiation process, and the like.

Accordingly, systems and methods for determining a sterilization time and sterilizing a vehicle interior based on the determined sterilization time are described. In at least some implementations, one or more sterilization factors, such as a first UV energy from sunlight, a second UV energy from a power UV source device, a time since the vehicle was last occupied, and/or an internal temperature of the vehicle interior may be analyzed to determine the sterilization time. As a result, at least some environmental factors, such as the first UV energy from sunlight, time since the vehicle was last occupied, and internal temperature may be factored into the determination of an appropriate sterilization time, where increases in any of these environmental factors may generally tend to reduce a sterilization time, during which the UV source device is operated to irradiate the vehicle interior with UV light. Moreover, the sterilization time may be communicated to a user device, such as a smartphone, and/or calculated by the user device itself, to facilitate display of the sterilization time and/or a variety of other status indicators for the convenience of the user. In at least some embodiments, the vehicle may be locked or otherwise secured to prevent entry by the user and/or other occupants during the sterilization time.

Exemplary embodiments of a system for sterilizing a vehicle are thus described above in detail. Although the systems herein described and illustrated in association with a motor vehicle, the disclosure is also intended for use on any means of conveyance including boats, aircraft, stationary objects, and other vehicles and objects capable of human occupation or human interaction. Moreover, it should also be noted that the components of the disclosure are not limited to the specific embodiments described herein, but rather, aspects of each component may be utilized independently and separately from other components and methods of assembly described herein.

This written description uses examples to disclose various embodiments, including the best mode, and also to enable any person skilled in the art to practice the various implementations, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A system for use in determining a sterilization time for sterilizing an interior of a vehicle, the system comprising:
a memory device; and
a processor configured to execute instructions stored in the memory device, which when executed, cause the processor to at least:
receive, from at least one detector device located within a vehicle interior, a first intensity of ultraviolet (UV) energy from sunlight incident on the at least one detector device;
determine, based at least in part on the first intensity of UV energy received from the detector device, a sterilization time for sterilizing the vehicle interior; and
in response to determining the sterilization time, transmit the sterilization time to a user device, wherein the user device is configured to display at least one of the sterilization time or a progress towards sterilization based on the sterilization time.

2. The system of claim 1, wherein the instructions, when executed, further cause the processor to at least:
determine a second intensity of UV energy emitted by a powered UV source device located within the vehicle interior; and
determine, based on the first intensity of UV energy and the second intensity of UV energy, the sterilization time for sterilizing the vehicle interior.

3. The system of claim 2, wherein the instructions, when executed, further cause the processor to at least:
control the powered UV source device to irradiate the vehicle interior for the sterilization time.

4. The system of claim 2, wherein the sterilization time is a function of a total intensity of UV energy within the vehicle interior, and wherein instructions, when executed by the processor, further cause the processor to at least:
increase the sterilization time, based upon a lookup table, in response to a decrease in at least one of the first intensity of UV energy and the second intensity of UV energy; and
reduce the sterilization time, based upon the lookup table, in response to an increase in at least one of the first intensity of UV energy and the second intensity of UV energy.

5. The system of claim 1, wherein the instructions, when executed, further cause the processor to at least:
determine an amount of time since the vehicle interior was last occupied; and
determine, based at least in part on the first intensity of UV energy received from the detector device and the amount of time since the vehicle interior was last occupied, the sterilization time for sterilizing the vehicle interior.

6. The system of claim 5, wherein the sterilization time is a function of a total intensity of UV energy within the vehicle interior and the amount of time since the vehicle interior was last occupied, and wherein instructions, when executed by the processor, further cause the processor to at least:
increase the sterilization time, based upon a lookup table, in response to a decrease in at least one of the first intensity of UV energy and the amount of time since the vehicle interior was last occupied; and
reduce the sterilization time, based upon the lookup table, in response to an increase in at least one of the first intensity of UV energy and the amount of time since the vehicle interior was last occupied.

7. The system of claim 6, wherein the instructions, when executed, further cause the processor to at least:
determine a second intensity of UV energy from a powered UV source device located within the vehicle interior; and
determine, based on the first intensity of UV energy, the second intensity of UV energy, and the amount of time since the vehicle interior was last occupied, the sterilization time for sterilizing the vehicle interior.

8. The system of claim 5, wherein the instructions, when executed, further cause the processor to at least:
determine a time since at least one system of the vehicle was last powered on to determine the time since the vehicle interior was last occupied.

9. The system of claim 1, wherein the instructions, when executed, further cause the processor to at least:
receive, from at least temperature sensor located within the vehicle interior, an internal temperature of the vehicle interior; and
determine, based at least in part on the first intensity of UV energy received from the detector device and the internal temperature of the vehicle interior, the sterilization time for sterilizing the vehicle interior.

10. The system of claim 1, wherein the detector device is one of a narrowband radiometer, a broadband radiometer, and a spectrophotometer.

11. The system of claim 1, wherein, in response to receiving the sterilization time, the user device is configured to display at least one of i) a countdown timer indicating a time until the vehicle interior is sterilized, ii) a status bar indicating a progress toward sterilization of the vehicle interior, and iii) an hourglass indicating that sterilization of the vehicle interior is occurring.

12. A system for sterilizing an interior of a vehicle, the system comprising:
at least one ultraviolet (UV) detector device disposed within the vehicle interior;
at least one powered UV source device disposed within the vehicle interior;
a memory device; and
a processor configured to execute instructions stored in the memory device, which when executed, cause the processor to at least:
receive, from the at least one detector device, a first intensity of UV energy from sunlight incident on the at least one detector device within a vehicle interior;
determine, based at least in part on the first intensity of UV energy received from the detector device, a sterilization time for sterilizing the vehicle interior; and
in response to determining the sterilization time, controlling the powered UV source device to emit UV energy within the vehicle interior for the sterilization time.

13. The system of claim 12, wherein the instructions, when executed, further cause the processor to at least:
determine a second intensity of UV energy from the powered UV source device disposed within the vehicle interior; and
determine, based on the first intensity of UV energy and the second intensity of UV energy, the sterilization time for sterilizing the vehicle interior.

14. The system of claim 13, wherein the sterilization time is a function of a total intensity of UV energy within the vehicle interior, and wherein instructions, when executed by the processor, further cause the processor to at least:
increase the sterilization time, based upon a lookup table, in response to a decrease in one or both of the first intensity of UV energy and the second intensity of UV energy; and
reduce the sterilization time, based upon the lookup table, in response to an increase in one or both of the first intensity of UV energy and the second intensity of UV energy.

15. The system of claim 12, wherein the instructions, when executed, further cause the processor to at least:
determine an amount of time since the vehicle interior was last occupied; and
determine, based at least in part on the first intensity of UV energy received from the detector device and the amount of time since the vehicle interior was last occupied, the sterilization time for sterilizing the vehicle interior.

16. The system of claim 12, wherein the instructions, when executed, further cause the processor to at least:
determine a time since at least one system of the vehicle was last powered on to determine the time since the vehicle interior was last occupied.

17. The system of claim 12, wherein the instructions, when executed, further cause the processor to at least:
receive, from at least temperature sensor located within the vehicle interior, an internal temperature of the vehicle interior; and
determine, based at least in part on the first intensity of UV energy received from the detector device and the internal temperature of the vehicle interior, the sterilization time for sterilizing the vehicle interior.

18. The system of claim 12, wherein the detector device is one of a UV detector, a narrowband radiometer, a broadband radiometer, or a spectrophotometer.

19. The system of claim 12, wherein the instructions, when executed, further cause the processor to transmit the sterilization time to a user device, wherein the user device is configured to display at least the sterilization time.

20. A method for determining a sterilization time for a vehicle interior, the method comprising:
receiving, by a processor and from at least one detector device located within the vehicle interior, a first intensity of ultraviolet (UV) energy from sunlight incident on the at least one detector device;
determining, by the processor and based at least in part on the first intensity of UV energy received from the detector device and a second intensity of UV energy emitted by a powered UV source device located within the vehicle interior, a sterilization time for sterilizing the vehicle interior; and
in response to determining the sterilization time, providing, by the processor, the sterilization time to a user device, wherein the user device is configured to display one of the sterilization time or a progress toward sterilizing the vehicle interior.
